SEQUENCE LISTING (2 sequences)

SEQ ID NO: 1 - DNA, 22 bp, Artificial Sequence
  Synthetic
tcaaggagtcaatttcgtaacg

SEQ ID NO: 2 - DNA, 19 bp, Artificial Sequence
  Synthetic
gttacgaatgacctccttg

(19) United States
(12) Patent Application Publication
(10) Pub. No.: US 2011/0039333 A1
(43) Pub. Date: Feb. 17, 2011

(54) DEFINED CONDITIONS FOR HUMAN EMBRYONIC STEM CELL CULTURE AND PASSAGE

(75) Inventors: Michael Kahn, Altadena, CA (US); Kouichi Hasegawa, Pasadena, CA (US); Jia-Ling Teo, Altadena, CA (US); Michael McMillan, Alhambra, CA (US)

Correspondence Address: DAVIS WRIGHT TREMAINE LLP/Los Angeles, 865 FIGUEROA STREET, SUITE 2400, LOS ANGELES, CA, 90017-2566, US

(73) Assignee: UNIVERSITY OF SOUTHERN CALIFORNIA, Los Angeles, CA (US)

(21) Appl. No.: 12/855,578

(22) Filed: Aug. 12, 2010

Related U.S. Application Data
(60) Provisional application No. 61/233,403, filed on Aug. 12, 2009.

(51) Int. Cl. C12N 5/0735

(52) U.S. Cl. 435/366; 435/404

(57) ABSTRACT

The invention relates to human pluripotent cells. More specifically, the invention provides a chemically defined xeno-free culture system that allows for long term expansion of human pluripotent cells. This culture system allows for human pluripotent cell lines to be maintained in the pluripotent state for an extended time while maintaining a normal karyotype and the ability to differentiate into all three germ layers.

### CROSS-REFERENCE TO RELATED APPLICATIONS

The present application claims the benefit of the filing date of U.S. Provisional application No. 61/233,403 filed Aug. 12, 2009, the disclosure of which is incorporated herein by reference in its entirety.

### FIELD OF INVENTION

This invention relates to stem cells. More specifically, the invention provides methods of culturing embryonic stem cell cultures and culture media useful therewith.

### BACKGROUND

Human embryonic stem cells (hESCs) are pluripotent cells capable of differentiation into all three embryonic germ layers (endoderm, mesoderm, and ectoderm) and are derived from preimplantation embryos. hESCs can be maintained in culture in an undifferentiated state for an extended period and can retain a normal karyotype, thereby potentially providing an unlimited supply of any cell type required for regenerative medicine (1-3). These cells have a wide variety of potential applications including study of human developmental biology, drug discovery and testing; and regenerative medicine. Differentiated hESCs can provide an unlimited and renewable source of therapeutic transplantable material. However, creating a source of this transplantable material requires establishing cell culture conditions that will maintain long term proliferative capacity, differentiation potential, and chromosome stability. A common and well-established technique of culturing embryonic stem cells requires the use of feeder cells (such as mouse fibroblasts) and animal serum (such as fetal bovine serum) for long-term maintenance of the pluripotent state. One drawback of these techniques is the batch to batch variability of using animal-derived products. Likewise, human-feeder-derived culture conditions suffer from lack of scalability and/or complicated culture media. Thus, there is a need to establish xeno-free culture conditions for therapeutic applications.

Historically, hESCs or induced pluripotent stem cells (iPSCs) are maintained on mouse embryonic feeder (MEF) layers and in media containing serum or serum replacement (1-3). These conditions include animal components and it has been reported that hESCs cultured under these conditions acquired a non-human mammalian sialic acid, Neu5Gc(25). Xenobiotic components associated with human pluripotent stem cell-derived cells utilized for transplantation likely will induce an immune response. Therefore, the development of a xeno-free hESC and iPSC culture system is required for regenerative medicine applications of human pluripotent cells. Several reports have described xeno-free culture systems using human feeders and human serum components (26-28). One example uses growth factor “cocktails” to establish a feeder-free and serum-free hESC culture system, comprising 15% scrum replacement, transforming growth factor β1 (TGFβ1), leukemia inhibitory factor, basic fibroblast growth factor, and fibronectin matrix (40). Further examples use human feeders with mouse embryonic fibroblast (MEF) conditioned medium (41). Other xeno-free systems utilize human serum and human feeders, (26). A few additional reports demonstrated xeno-free and feeder-free culture systems for the maintenance of the undifferentiated state of hESC (4-8). These culture systems are quite complex requiring a large number of human protein components and/or GMP-certified animal components. Eventual therapeutic applications of hESC will require the development of scalable, bulk expansion techniques unencumbered by the use of complicated protein mixture or supplementation from other biological sources. Furthermore, defined conditions that eliminate the risk of zoonotic disease transmission or limit undesirable variability in preparations. Therefore, it would be highly beneficial to develop a simple chemically defined pluripotent stem cell culture system (29-31). To this end, several reports have appeared describing the screening of large chemical libraries but, to this point, these efforts have not led to a vastly simplified xeno-free culture media.

Although several feeder- and xeno-free culture conditions have been reported (4-8), the conditions that are required comprise complex culture medium or many human derived components. In order to meet good manufacturing practice (GMP) standards, replacing such components with small molecules would provide significant advantages, However, this is contingent upon the understanding of the key signaling pathways involved in hESC self-renewal.

The β-catenin protein and co-activators CBP and p300 play important roles in hESC self-renewal and differentiation. One example of a signaling pathway involving β-catenin, CBP, and p300 is the Wnt signaling pathway, which has been demonstrated to maintain pluripotency in stem cells under certain conditions (36, 37) and is critical for the expansion of progenitors (38). Wnt signaling has also been demonstrated to be important for the maintenance of pluripotency in both mouse and human embryonic stem cells in cultured (11). Expression of multiple components of the Wnt pathway is evident in the P19 human embryonal carcinoma cell lines, as well as in embryonic stem cells (39).

Recent work has demonstrated that β-catenin/CBP signaling is critical for cell proliferation without differentiation, whereas a switch to β-catenin/p300 is critical to initiate differentiation and limits proliferation (18). The Wnt/β-catenin pathway normally regulates expression of a range of genes involved in promoting both proliferation and differentiation. Activation of the Wnt pathway allows β-catenin to accumulate in the nucleus, bind to members of the T-Cell Factor (TCF) family of transcription factors, and form a transcriptionally active complex, by recruiting either the transcriptional coactivator CBP (Creb-binding protein) or its closely related homolog, p300.

Small molecules, such as IQ-1, as reported by Miyabashi and colleagues (18), bind to the PR 72/130 subunit of the serine/threonine phosphatase PP2A. The binding of IQ-1 to PR72/130 leads to decreased phosphorylation of the coactivator protein p300 at Ser-89. Since the phosphorylation of p300 at Ser-89 enhances the binding affinity of β-catenin to p300, inhibitors or small molecule Wnt signaling modulators, such as IQ-1 thereby diminish the β-catenin/p300 interaction and prevent β-catenin coactivator switching from CBP to p300. Without being bound by a specific mechanism, the invention is based on the premise that the increase in β-catenin/CBP mediated transcription at the expense of the β-catenin/p300 interaction is critical for the maintenance of pluripotency.

The inventors previously demonstrated that small molecule Wnt signaling modulation allows for long term Wnt mediated expansion of mouse ESCs (mESCs) (18). However, hESC appear to share very few characteristics with their mouse counterparts, besides the key “pluripotency transcription factors” i.e. Oct4, Nanog and Sox2, which they have in common. The extrinsic factors regulating hESC main and early differentiation events seem to differ from mESC and to date are poorly understood.

The inventors previous, investigations demonstrated that small molecule modulators of the Wnt signaling cascade can maintain mESC proliferation and pluripotency for extended periods of time in the absence of serum (18, 21). Furthermore, gene expression differences between parental “weak” hESC and culture-adapted “strong” hESC sublines further validated the importance of Wnt signaling in hESC self-renewal. Based upon earlier reports, Wnt signaling has been demonstrated to have positive effects on the maintenance of mESCs, and also appears to facilitate the maintenance of the undifferentiated phenotype and maintains expression of critical “pluripotency” transcription factors in hES cells. Brivanolou and colleagues described the positive, although limited-time frame effects of BIO (6-bromoindirubin-3′-oxime), a known small molecule inhibitor of the Ser/Thr kinase GSK-3, a key Wnt signal regulator, on both mouse and human ESC maintenance, while preserving a normal differentiation program capacity after withdrawal (11). There has been significant controversy regarding this report (12). However, selective modulation of coactivator usage in the Wnt signaling cascade allows for long term ESC pluripotency.

### SUMMARY OF THE INVENTION

In an embodiment, the invention includes a culture medium for maintaining proliferation and pluripotency of human embryonic stem cells (hESCs), the culture comprising: a basal medium; basic fibroblast growth factor; Wnt3a protein; and a small molecule Wnt signaling modulator. The basal medium may comprise DMEM/F-12. The small molecule Wnt signaling modulator maybe ID-8, IQ-1, ICG-427, BIO, LiCl, a combination thereof, a small molecule structurally related to ID-8, IQ-1, ICG-427, BIO, LiCl, or a pharmaceutical equivalent, analog, derivative, salt or prodrug of any of the foregoing or any molecule that modulates Wnt signaling in order to maintain hESCs in ah undifferentiated state. The culture medium may further comprise Insulin-Transferrin-Selenium (ITS).

In another embodiment, the invention includes a method of culturing pluripotent human embryonic stem cells (hESCs), comprising: providing a quantity of hESCs; placing the quantity of hESCs in a culture vessel coated with an extracellular matrix; and culturing the quantity of hESCs with a culture medium comprising a basal medium, basic fibroblast growth factor, Wnt3a protein, and a small molecule Wnt signaling modulator, wherein the hESCs remain pluripotent and proliferate. The basal medium may comprise DMEM/F-12. The culture medium may further comprise Insulin-Transferrin-Selenium (ITS). At least a portion of the quantity of hESCs may be maintained for more than 17 passages. At least a portion of the quantity of hESCs may be capable of differentiating into all three germ layers after the culturing step.

In another embodiment, the invention includes a method of passaging a quantity of pluripotent human embryonic stem cells (hESCs), comprising: providing the quantity of hESCs in a first quantity of a culture medium comprising a basal medium, basic fibroblast growth factor, Wnt3a protein, and a small molecule Wnt signaling modulator; adding a disintegrin and GRGDTP peptides to the quantity of hESCs; allowing the quantity of hESCs to detach from the dishes; rinsing the detached quantity of hESCs in a second quantity of the culture medium; and seeding at least a portion of the quantity of hESCs on new dishes. The basal medium may comprise DMEM/F-12. The small molecule Wnt signaling modulator maybe ID-8, IQ-1, ICG-427, BIO, LiCl, a combination thereof, a small molecule structurally related to ID-8, IQ-1, ICG-427, BIO, LiCl, or a pharmaceutical equivalent, analog, derivative, salt or prodrug of any of the foregoing or any molecule that modulates Wnt signaling in order to maintain hESCs in an undifferentiated state. The culture medium may further comprise Insulin-Transferrin-Selenium (ITS). The quantity of hESCs may be provided on Fibronectin-Laminin-Vitronection (FLV)-coated dishes. The disintegrin may be Echistatin. At least a portion of the quantity of hESCs cells may be passaged by repeating the method steps. The method steps may be repeated more than 17 times. The hESCs may be capable of differentiating into all three germ layers after the seeding step. The seeding may further comprise seeding in a 1 to 2 ratio.

In another embodiment, the invention includes a composition comprising human embryonic stem cells (hESCs) and a culture medium, comprising a basal medium, basic fibroblast growth factor, Wnt3a protein, and a small molecule Wnt signaling modulator. The basal medium may comprise DMEM/F-12. The small molecule Wnt signaling modulator may be ID-8, IQ-1, ICG-427, BIO, LiCl, a combination thereof, a small molecule structurally related to ID-8, IQ-1, ICG-427, BIO, LiCl, or a pharmaceutical equivalent, analog, derivative, salt or prodrug of any of the foregoing or any molecule that modulates Wnt signaling in order to maintain hESCs in an undifferentiated state. The culture medium may further comprise Insulin-Transferrin-Selenium (ITS).

The above-mentioned and other features of this invention and the manner of obtaining and using them will become more apparent, and will be best understood, by reference to the following description, taken in conjunction with the accompanying drawings. The drawings depict only typical embodiments of the invention and do not therefore limit its scope.

### BRIEF DESCRIPTION OF THE FIGURES

FIG. 1. Gene expression analysis of hESC subline cells. (A) Gene number of up regulated (>2.0 fold) or (B) down regulated (<0.5 fold) only in subline 1, subline 2 and commonly in both cell line (merged region) compare to parental cells. (C) Gene list of commonly up regulated and (D) down regulated in both subline cells.

FIG. 2. Chromosomal distribution of hESC subline cells. (A) Graphical representation of the chromosomal distribution of the 227 genes up regulated in subline 1. (B) Graphical representation of the chromosomal distribution of the 67 genes up regulated in subline 2. (C) Graphical representation of the chromosomal distribution of the 265 genes down regulated in subline 1. (D) Graphical representation of the chromosomal distribution of the 177 genes down regulated in subline 2.

FIG. 3. A combination of ID-8 and Wnt ligand enhances hESC self-renewal. (A) Screening of small molecule Wnt signaling modulator in clonal proliferation assays with HES2 cells in MEF-CM. (B) Colony numbers par seeded cell numbers indicates as a replating efficiency. Cultured plates stained with ALP and (C) morphologies of typical colonies at day 7. (D) ALP staining and OCT4 immunostaining of HES2 cells cultured in MEF-CM with 0.5 μM ID-8 and 100 ng/ml Wnt3a. (E) Replating efficiency of HES3 and H9 cells cultured in MEF-CM with or without and Wnt3a. Quantitative PCR analysis of pluripotent markers (F) and differentiation markers (G and H) in hESC cultured in MEF-CM with or without ID-8 and Wnt3a. All scale bar indicates 200 μm and all error bar indicates standard deviation. (I) Molecular structure of IQ-1. (J) Molecular structure of ICG-001. (K) Molecular structure of ICG-427.

FIG. 4. Identification of ID-8 target molecule. (A) Molecular structure of ID-8. (B) Silver staining of proteins pulled down with biotin-conjugated ID-8 and streptavidin-sharose in SDS-page. Arrow indicates a specific band (60 kDa) decreased by competition with non-biotin conjugated ID-8. (D) Western blotting with DYRK family specific antibodies. Samples were pulled down with resin only, biotin-conjugated ID-8 or biotin-conjugated ID-8 plus non-conjugated ID-8. (D) Western blotting with DYRK antibodies after transient transfection of DYRK miRNA or control scramble miRNA expression plasmid. (E) Western blotting of stable transfectants. (F) Clonal proliferation assays of H9 hESC stably transfected with miRNA.

FIG. 5. Xeno-free, feeder-free hESC culture system using ID-8 and Wnt activation. Images of undifferentiated hESCs under three media conditions: (A) 0.2 μM CHIR90021 plus bFGF; (B) 1.0 μM Kenpaullone; (C) 1.0 μM Kenpaullone plus bFGF, and (D) 0.5 μM Kenpaullone plus bFGF.

FIG. 6. Characterization of human pluripotent cells maintained in the xeno-free and feeder-free culture system. (A) Typical colony morphologies at passage 7. (B) Immunostaining of undifferentiated cell markers at passage 17. (C) G-banding karyotype at passage 22. (D) Immunostaining of ectoderm (βIII-Tubulin and GFAP), mesoderm (aSMA, Desmin), endoderm (GATA6 and AFP) and definitive endoderm (HNF1b)-derived cell markers in in vitro differentiation assay after passage 17. (E) Ectodermal (neuroepithelium and pigmented cell), mesodermal (muscl and cartilage) and endodermal (gut-like and lung-like) tissue in teratoma formed with hESC cells after passage 22 in the culture system. All scale bars indicate 100 μm.

### DESCRIPTION OF THE INVENTION

The application of hESC in regenerative medicine will require the propagation of large numbers of cells in the absence of animal products (xeno-free conditions). Most xeno-free hESC culture systems require human feeder cells and/or highly complicated culture media. Likewise, these culture systems may not be used for GMP manufacturing of hESC or iPSC, which is an important step towards future regenerative medical therapies.

After investigating the signaling cascades involved in the enhancement of hESC survival and/or self-renewal, the inventors have developed a simple, chemically defined culture method. In one embodiment, the invention provides a chemically defined xeno-free culture system that allows for long term expansion of human pluripotent cells, which meet GMP standards. These culture conditions need not, and in various embodiments do not, include complicated supplements, serum, serum replacement or albumin. Using this culture system, several human pluripotent cell lines can be maintained in the pluripotent state for an extended time (>20 passages) with normal karyotype and the ability to differentiate into all three germ layers.

One embodiment of the present invention provides a human pluripotent stem cell culture system comprising minimal growth factors and small molecule Wnt signaling modulators, and which does not include feeder cells, animal serum, and xenogenic ingredients. Additionally, a mild passaging system that utilizes a synthetic disintegrin peptide is provided. The system requires purified human extracellular matrix molecules, which can be certified for GMP.

One skilled in the art will recognize many methods and materials similar or equivalent to those described herein, which could be used in the practice of the present invention. The present invention is in no way limited to the methods and materials described. For purposes of the present invention, the following terms are defined below.

The term “stem cell” refers to an undifferentiated cell which is capable of self-renewal, i.e., proliferation to give rise to more stem cells, and may give rise to lineage committed progenitors which are capable of differentiation and expansion into a specific lineage. The stem cell refers to a generalized mother cell whose descendants (progeny) specialize, often in different directions, by differentiation, e.g., by acquiring completely individual characters, as occurs in progressive diversification of embryonic cells and tissues.

As used herein, the term “human embryonic stem cells (hESCs)” refers generally to embryonic, of human, origin stem cells. hESCs can be expanded into all three embryonic germ layers including gut epithelium (endoderm), cartilage, bone, smooth muscle, and striated muscle (mesoderm), and neural epithelium, embryonic ganglia, and stratified squamous epithelium (ectoderm). Embryonic stem cells are derived from the inner cell mass of preimplantation embryos.

“Pluripotent” as used herein refers to stem cells that have the potential to differentiate into any of the three germ layers: endoderm, mesoderm, or ectoderm. Pluripotent stem cells can give rise to any fetal or adult cell type cells that are capable of indefinite proliferation in vitro while remaining undifferentiated and maintaining a normal karyotype throughout long-term culture.

“Induced pluripotent stem cells” (iPS) cells are a type of pluripotent stem cell artificially derived from non-pluripotent cells, typically an adult somatic cell, by inducing a “forced” expression of certain genes.

The term “undifferentiated” as used herein refers to pluripotent embryonic stem cells which have hot developed a characteristic of a more specialized cell. As will be recognized by one of skill in the art, the terms “undifferentiated” and “differentiated” are relative with respect to each other. An embryonic cell which is “differentiated” has a characteristic of a more specialized cell. Differentiated and undifferentiated cells are distinguished from each other by several well-established criteria, including morphological characteristics such as relative size and shape, ratio of nuclear volume to cytoplasmic volume; and expression characteristics such as detectable presence of known markers of differentiation.

Any of various types of differentiated cells may be obtained by inducing differentiation of a substantially homogeneous population of embryonic stem cells produced according to methods of the present invention. Examples of such differentiated cells obtained according to methods of embodiments of the present invention illustratively include committed neuronal precursors, neurons* committed pancreatic beta cell precursors and pancreatic beta cells, bone cell precursors, bone cells, liver cell precursors, liver cells, muscle cell precursors, muscle cells, cardiac muscle precursors, cardiac muscle cells, skin cell precursors, skin cells, kidney cell precursors, kidney cells, vascular endothelial cell precursors, vascular endothelial cells, blood cell precursors, blood cells, adipose cell precursors, and adipose cells.

“Small molecule Wnt signaling modulators” as used herein refers to molecules that modulates Wnt signaling in order to maintain the undifferentiated state of hESCs and promote proliferation. This maybe accomplished by one of several ways, including: 1) diminishing the β-catenin/p300 interaction, which prevents the β-catenin coactivator switching from CBP to p300, thereby increasing the β-catenin/CBP complex; 2) inhibiting GSK-3, a key Wnt signal regulator; or 3) binding CBP and inhibiting β-catenin/TCF-mediated transcription. Small molecule Wnt signaling modulators that can be used in accordance with the present invention may include, but are not limited to, for example, ID-8, IQ-1, ICG-427, BIO, LiCl, a combination thereof, a small molecule structurally related to ID-8, IQ-1, ICG-427, BIO, LiCl, or a pharmaceutical equivalent, analog, derivative, salt or prodrug of any of the foregoing or any molecule that modulates Wnt signaling in order to maintain hESCs in an undifferentiated state.

Further, small molecule Wnt signaling modulators are classified into 2 groups: 1) those that activate Wnt signaling and 2) p300/catenin antagonists. Wnt activators are Wnt3a or GSK-3 inhibitors, including but not limited to BIO or LiCl. p300/catenin antagonists diminish β-catenin/p300 interaction and include but are not limited to ID-8, IQ-1, ICG-427.

### EXAMPLES

The following examples are intended to illustrate, but not to limit, the scope of the invention. While such examples are typical of those that might be used, other procedures known to those skilled in the art may alternatively be utilized. Indeed, those of ordinary skill in the art can readily envision and produce further embodiments, based on the teachings herein, without undue experimentation.

### Example 1

### Cells and Cell Culture

The hESC lines HES2, HES3, H1 and H9, and the human fetal dermal fibroblasts-derived iPSC line were maintained using standard cell culture methodology. For enzymatic bulk expansion, the cells were cultured on mitotically arrested mouse embryonic fibroblast (MEF) feeder cell layers in DMEM/F12 supplement with 20% Knockout serum replacement (Invitrogen, CA), L-glutamine, non-essential amino acid and 4 ng/ml recombinant FGF2 (Peprotech), as described previously. For feeder-free culture, the MEF-feeder was removed by sedimentation in passage, and cells were cultured in MEF-conditioned medium on 30-fold diluted Matrigel (BD Biosciences, CA)-coated culture dishes.

The clonal proliferation assay was described previously. Briefly, the hES cells in feeder-free culture were dissociated completely with 0.05% trypsin-EDTA.(Invitrogen), and are seeded at 10⁴ cells/well in Matrigel-coated 6-well culture plates and cultured in MEF-conditioned medium. Various concentration of Wnt3a (purified in our laboratory or purchased from Peprotech), IQ-1, ID-8 and/or ICG-001 (all three chemicals were synthesized in our laboratory), were supplemented into the culture media at the onset of seeding, then continuously until the end of culturing. For all assays, the cell and colony morphology were examined under microscope, and the replating efficiency was examined by counting the number of colonies after 7 days of culture.

For xeno-free, feeder-free culture, the cells were passaged into 2 μg/cm² human Fibronectin (Sigma), 10-20 μg/cm² human Laminin (Sigma) and 10 μg/cm² human Vitronectin (Invitrogen)-coated culture dishes and cultured in DMEM/F-12 supplement with L-glutamine, a non-essential amino acid, ITS, 20-25 ng/ml Wnt3a and 500 nM ID-8, and with or without 4 ng/ml FGF2. The culture medium was changed daily. Some of colonies became detached from the edge region 5 to 7 days after culturing, and then cells were treated with 0.5 mg/ml GRGDTP peptides (AnaSpec) or 1 μg/ml Echistatin (Sigma) and incubated at 37° C. for 15-45 minutes until almost all of the colony starts to detach from the edge region. The colonies were detached and partially dissociated into clumps by gentle pipetting, and then the clumps were washed with culture medium, collected by centrifugation and seeded on new dishes as 1 to 2 split ratio.

### Example 2

### Microarray and Quantitative PCR Analysis

Each 3 samples of parental KhESC-1, subline-1 and -2 were cultured in feeder-free condition and total RNA were purified with Trizol (Invitrogen) followed by RNeasy mini kit (Qiagen): Total 9 samples of 4 μg RNA were labeled with one cycle target labeling and hybridized with Human Genome U133 plus 2.0 Array (Affimetrix) respectively in accordance with the manufacture's protocol. The gene expression data were analyzed using GeneSpring GX software (Agilent), and the gene lists of up (>2.0-fold) or down (<0.5-fold)-regulated genes in subline cells from parental KhES-1 were made with criterion of t-test p-value <0.05 and 1-way ANOVA p-value <0.05 after removing Flagged spots.

For quantitative PCR analysis, total RNA was isolated from hESCs cultured in feeder-free condition with or without Wnt3a and Id-8 using RNeasy micro kit (Qiagen). RT was performed with Omniscript (Qiagen) and quantitative RT-PCR was then performed, using each gene-specific primer/probe mix (TaqMan Gene Expression Assays), TaqMan 2× Master Mix, and ABI PRISM 7900 Sequence Detection system (Applied Biosystems, CA) according manufacture's protocol. The PCR data was analyzed by delta/delta OT method and normalized with PPIA expression.

### Example 3

### Identification of ID-8 Target

The cells were washed twice with 5 mL of ice-old PBS and scraped using 1 mL of PBS containing protease inhibitor cocktail (Calbiochem 539137). The cells were collected by centrifugation and the cell pellet was frozen and stocked with liquid nitrogen. Total 2 g of cell pellet was thawed on ice and then suspended in 20 mL of chilled M-Per (Pierce 78501) buffer containing 1 mM DTT and the protease inhibitor cocktail. The cell suspension was mixed and lysed on a nutator (BD Diagnostics) for 30 minutes at 4° C. The lysate was then centrifuged at 10,000 rpm for 30 minutes at 4° C., and the supernatant was divided into two equal aliquots in 50 mL centrifuge tubes. DMSO control was added to one tube and 100 μM ID-8 was added to the other tube. The tubes were incubated for 1 hour on a nutator at 4° C. At the same time, 400 μl of 50% slurry of streptavidin-sepharose (GE Healthcare 17-5113-01) was equilibrated in M-Per buffer and then added to 10 mL of 50 μM biotinylated ID-8 and incubated for 1 hour at 4° C. The sepharose beads were then washed extensively with M-Per to remove unbound biotinylated ID-8.

After pre-treatment with free competitor ID-8 or DMSO control, the streptayidin-sepharose bound with biotinylated ID-8 was applied on the lysates. The mixture was then incubated on a nutator for 4 hours at 4° C. The sepharose beads, containing proteins bound to biotinylated ID8 were washed three times with 1 mL of M-Per buffer, and then mixed with 2× Laemmli Sample Buffer and boiled for 5 minutes. The Sample Buffer was separated from the sepharose using illustra MicroSpin Columns (GE Healthcare 27-3565-01). Bound proteins eluted in the Laemmli Buffer were subjected to SDS-PAGE and stained by silver staining (Invitrogen LC6070). The specific bands lost by competitor free ID-8 was analyzed by LC/MS (LTQ-XL, Thermo Scientific).

The elution was also analyzed by or western blotting with specific antibodies against DYRK1a (Cell Signaling 2771), DYRK2 (Abeam ab37912), DYRK3 (Santa Cruz sc-66868), DYRK4 (Abeam ab37911), or HIP2K (Santa Cruz sc-100383).

### Example 4

### Knockdown Assay

The several DYRK miRNA were designed and lentiviral vector plasmids were constructed with Block-iT Pol II miR RNAi kit (Invitrogen) by according to the manufactures protocol. The knockdown efficiency of miRNA expression plasmids were evaluated by transient transfection and western blotting. Then most efficient miRNA sequence; antisense 5′-tcaaggagtcaatttcgtaacg-3′ and sense, 5′-gttacgaatgacctccttg-3′; was used for further lentivirus production.

To produce the miRNA expression lentiviral vectors, the plasmid was co-transfected with packaging plasmids for vesicular stomatitis virus (VSV) G protein pseudo-typed virus into 293T cells with Fugene6 Transfection reagent (Roche). Viral supernatants were collected and concentrated by ultracentrifugation for 2 hours. Then feeder-removed H9 cells were infected with the virus in MEF-CM supplemented with 10 μg/ml polybrene. The infected cells were selected and isolated as pools by 500-1000 μg/ml Blasticidin for 1 week. The infected H9 cell pools were evaluated by western blotting and then used for the clonal proliferation assay.

### Example 5

### Characterization, Karyotype Analysis and Differentiation Assay

The hESC colonies were fixed with 4% paraformaldehyde, and alkaline phosphatase (ALP) activity was determined with a Vector Blue Alkaline Phosphatase Substrate Kit (Vector Laboratories, CA). Immunostaining was carried out with the following primary antibodies: TRA-1-60 and TRA-1-80 (Santa Cruz Biotechnology, CA), anti-stage specific embryonic antigen (SSEA)-3 and -4 (Developmental Studies Hybridoma Bank at the University of Iowa), anti-OCT-3 (clone C-10, Santa Cruz Biotechnology, CA), or goat anti-Nanog (R&D systems, MN). Then, samples were incubated with Alexa Fluor 488- or 594-conjugated (Molecular Probes, NY), then detected with indirect immunofluorscence microscopy.

For karyotyping, hES cells were treated with 100 ng/ml colcemid (KaryoMax, Invitrogen), dispersed and fixed. Fifty to 20 cells at metaphase in each sample were analyzed for G-banding at 300 to 500 band levels by trypsinize and Giemza's staining.

For differentiation assays, embryoid bodies (EB) were formed for 2 -weeks in low-attachment culture plates and then plated onto glass culture slides as described previously. After the cells grew out from attached EBs, they were fixed with 4% PFA, incubated with anti-N-tubulin (clone TU-20, Chemicon International, CA), -GFAP (clone 4A11, BD biosciences, CA), -alpha smooth muscle actin (clone 1A4, Dako Cytomation, Denmark), -Desmin (clone Ab-1, Thermo Scientific, MA), -GATA6 (clone 222228, R&D systems, MN), -HNF1b antibody (goat polycronal, Santa Cruz Biotechnology, CA) or -AFP antibody (clone C3, Sigma-Aldrich, MO) as the primary antibodies and then detected using Alexa Fluor 488- or 4594-conjugated secondary antibodies (Molecular Probes, NY) and fluorescence microscopy.

For teratoma formation, hES cells were dissociated as clumps and 200 μl suspension containing approximately 10⁷ cells were subcutaneously injected into SCID mice (Jackson Laboratories). After 2 to 3 months, the resulting teratomas were dissected and fixed with Bouin's fixative solution. Samples were embedded in paraffin, sectioned at 5 μm thickness, and subsequently stained with hematoxylin and eosin (HE-staining).

### Example 6

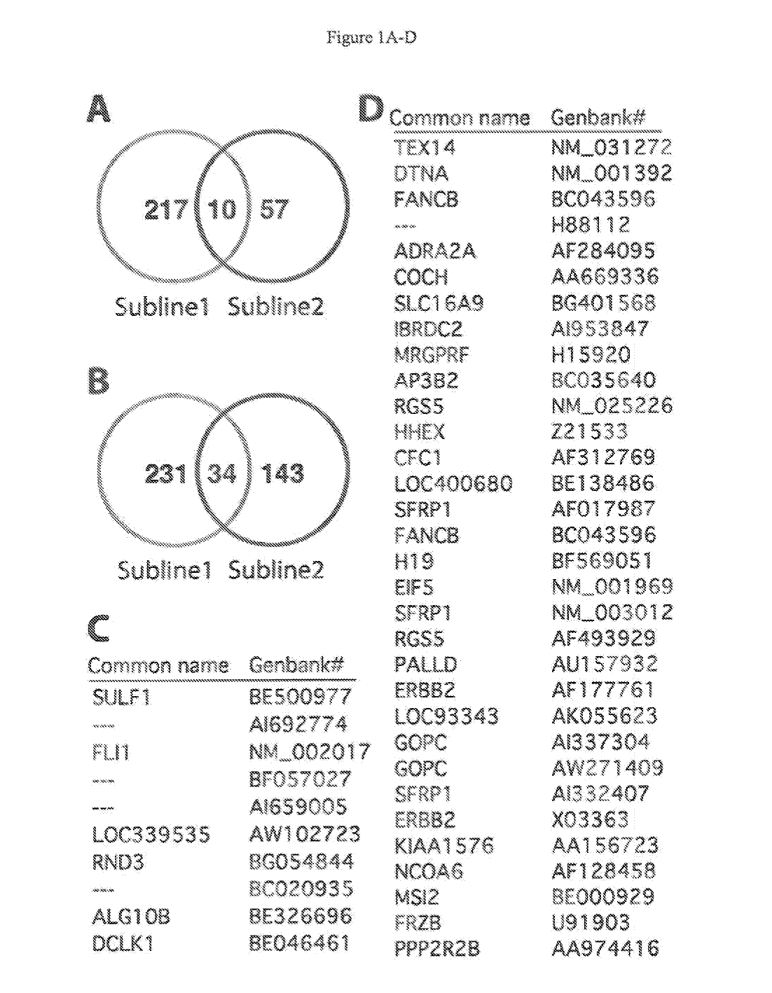

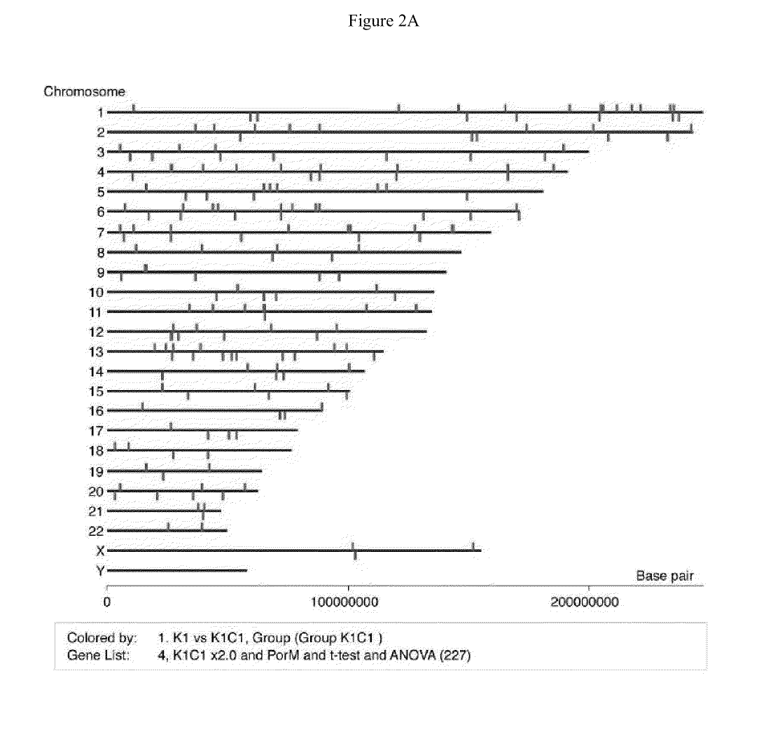

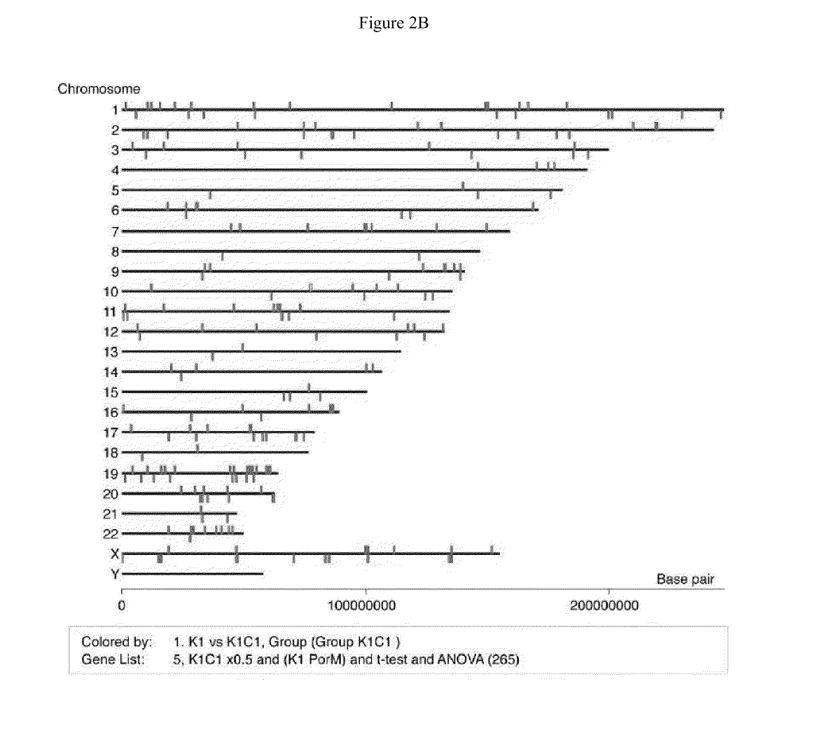

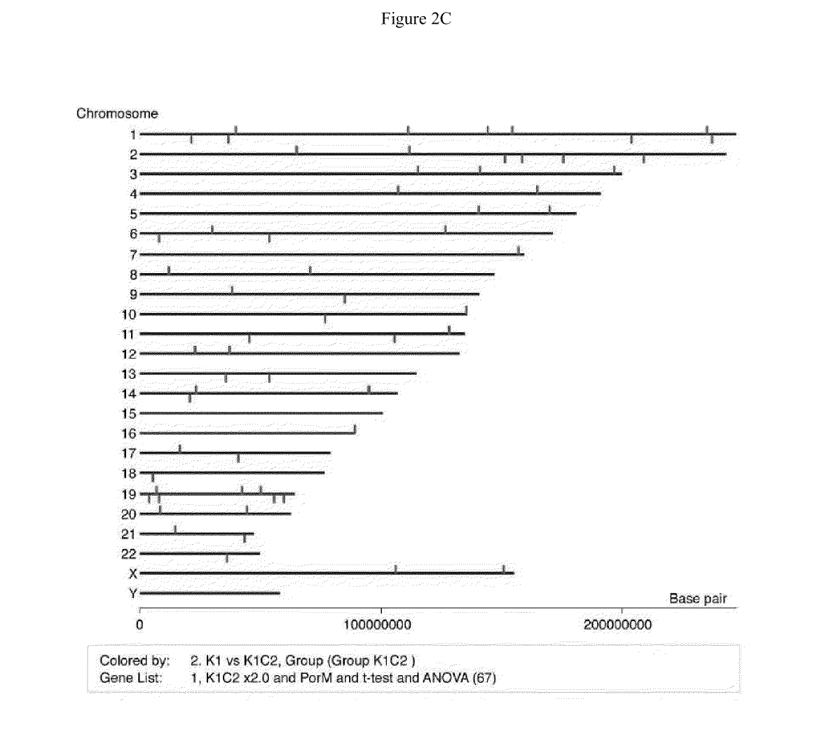

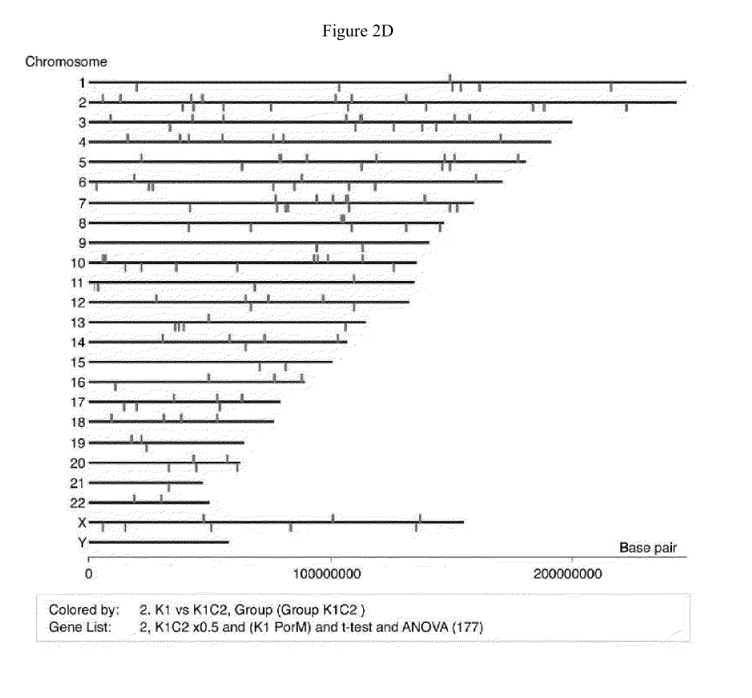

To explore signaling pathways that enhance hESC self-renewal, the inventors compared gene expression profiles between parental hESC and two hES subline cells, which were tolerant of complete dissociation, proliferating robustly, yet exhibiting a normal genotype (9). cDNA microarray analysis showed 227 and 67 gene probes were up regulated (>2.0 fold) and 265 and 177 genes were down regulated (<0.5 fold) in sublines 1 and 2, respectively (FIG. 1 A,-B). The up-or down regulated genes were widely distributed over all of the chromosomes and there were no specific genomic regions in which these genes were concentrated (FIG. 2). Interestingly, only 10 genes were significantly upregulated in both sublines. Included in this group was the gene RND3, which is reported to bind and inhibit ROCKI function (FIG. 1 C). This may contribute to the sublines tolerance of single cell dissociation as ROCK inhibition is known to enhance hESC survival (10). 34 genes were down regulated in both hESC sublines, including a number of Wnt inhibitory molecules, e.g. SFRP1 and FRZB (FIG. 1 D). This data suggested that modulation of Wnt signaling might be beneficial to hESC maintenance.

The canonical Wnt/β-catenin signaling has been reported to be involved in hESC survival, proliferation and also differentiation (9, 11-15), however, a rationale for the dichotomous behavior of Wnt/β-catenin signaling in controlling both proliferation and differentiation of hESC has been unclear. Utilizing a chemical genomic approach, (16, 17), the inventors have previously developed a model that rationalizes these divergent behaviors based upon differential coactivator usage (16, 18, 19). The inventors previously demonstrated that the small molecule IQ-1, in conjunction With Wnt3a, was sufficient to maintain mESC proliferation and pluripotency for extended periods of time in the absence of serum by enhancing Wnt/β-catenin/CBP-mediated self-renewal signaling and preventing the switch to Wnt/β-catenin/p300-mediated differentiation (18). These data supported the proposal that modulation of Wnt signaling by small molecules might be beneficial to hESC maintenance.

### Example 7

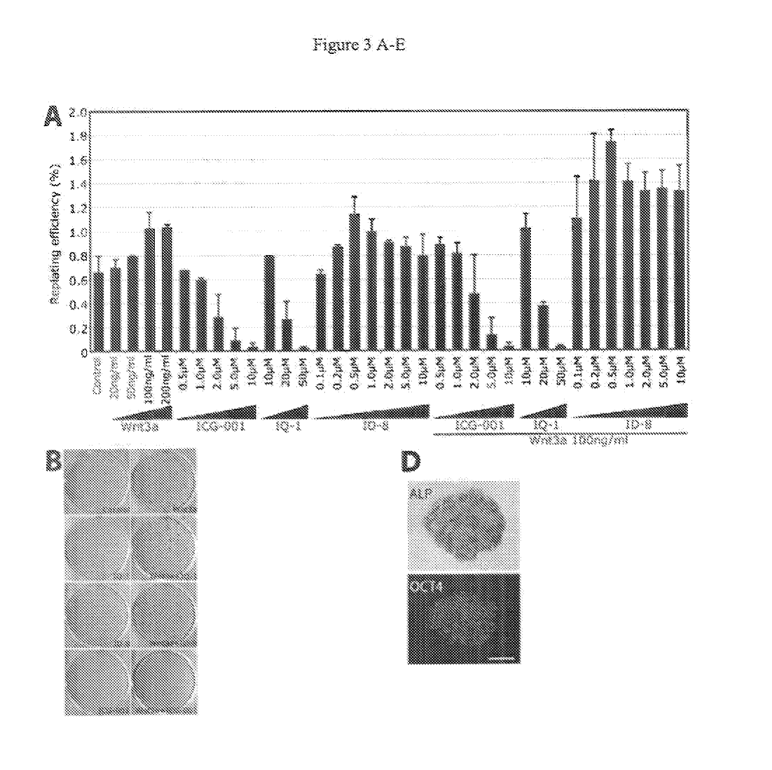

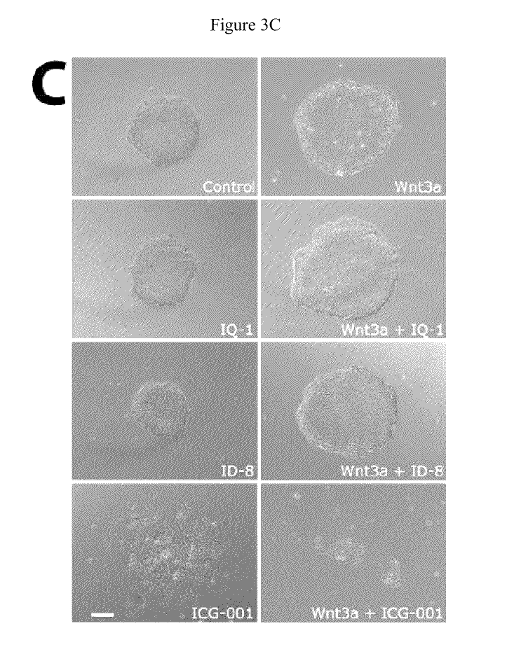

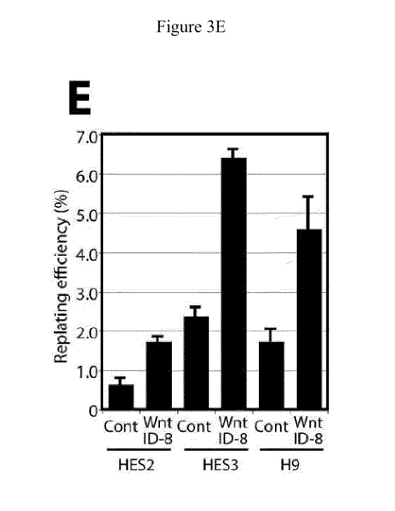

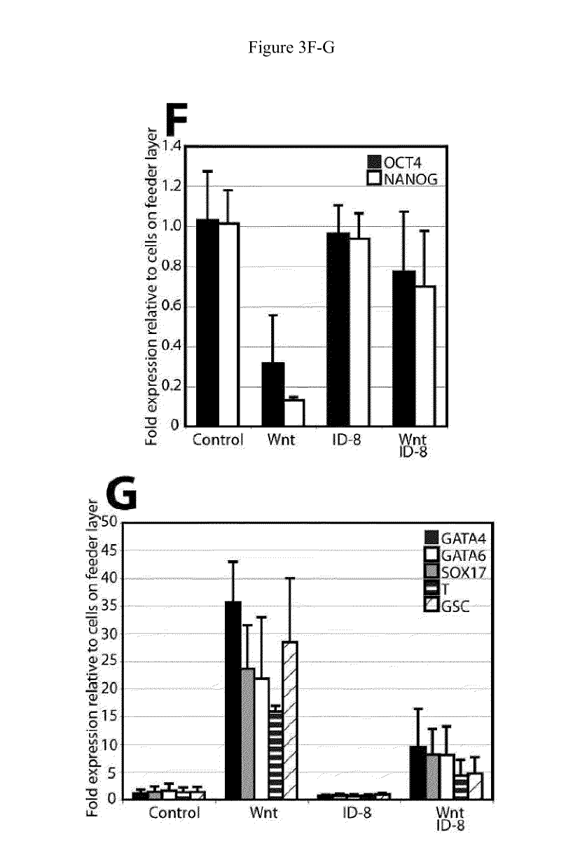

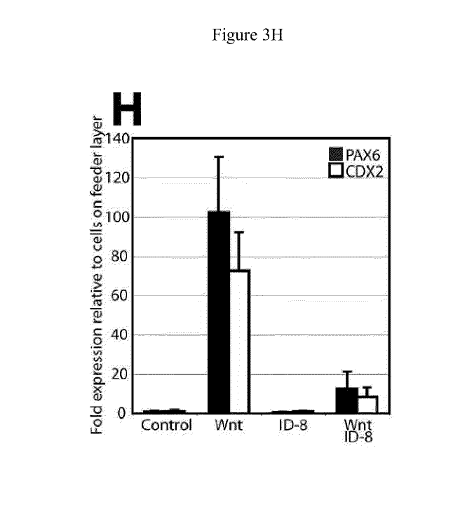

The effects of canonical Wnt ligand, Wnt3a and several small molecule Wnt signaling modulators in a clonal proliferation assay were investigated first (9) (FIG. 3A). When HES2 cells (2) were dissociated into single cells and seeded on Matrigel-coated plates in mouse embryonic fibroblast-conditioned medium (MEF-CM)(20), the colony forming efficiency was ˜0.6% (replating efficiency: colony number/seeded cell number). Supplementation with Wnt3a enhanced colony formation (˜1% at 100 ng/ml). The colonies were larger and consisted of more cells. However, the cells in the colonies appeared flatter than with the control non-Wnt conditions (FIG. 3B-C). these results suggested, that Wnt3a supplementation enhances hESC survival and proliferation but also induces differentiation. Supplementation with ICG-001, which enhances the β-catenin/p300 association, strongly induced cell differentiation and death in the presence or absence of Wnt. IQ-1, which enhances the β-catenin/CBP association, did not significantly affect hESC self-renewal by itself, and somewhat prevented Wnt-induced differentiation. ID-8, which was identified in the same screen as IQ-1 (21), also enhances the β-catenin/CBP association and increased hESC survival (˜1.1% at 0.5 μM). The combination of Wnt3a and ID-8 further increased survival (˜1:7%) and completely prevented Wnt-induced differentiation without disrupting proliferation. These cells expressed pluripotency markers i.e. Alkaline Phosphatase (ALP), OCT4 and NANOG at essentially the same levels as control hESCs, and repressed Wnt-induced differentiation gene expression, i.e. GATA6, SOX17, T (Brachury) and CDX2 (FIG. 3D and F-H). To examine the scope of this effect in hESC, clonal proliferation assays were performed with the additional hESC lines, HES3 (2) and H9 (1) (FIG. 3H). In both, the combination of Wnt and ID-8 enhanced hESC self-renewal.

### Example 8

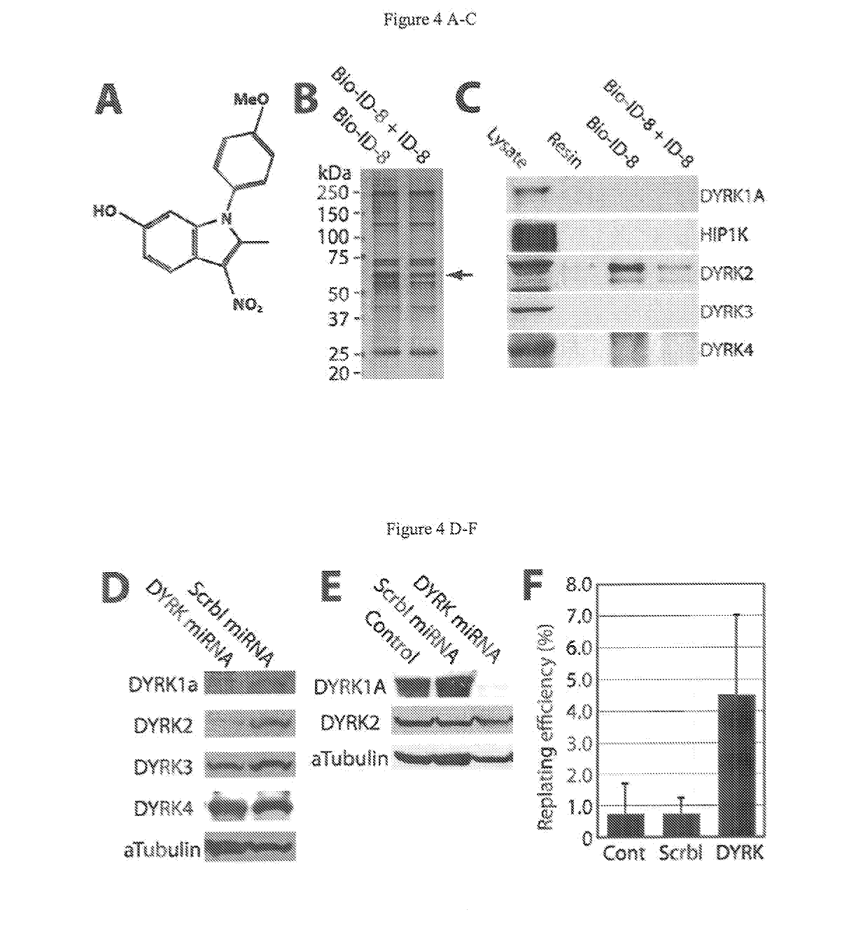

ID-8 (FIG. 4A) can enhance β-catenin/CBP-mediated transcription, however, its direct molecular target(s) remained unidentified. To identify the target(s) of ID-8, the inventors utilized affinity chromatography (FIG. 4B). The inventors identified the dual specificity kinase DYRK2 from cell lysates and its binding to the ID-8 affinity column was competed away by free ID-8. Western blotting of the precipitated proteins identified not only DYRK2 but also DYRK4 (FIG. 4C). These data suggested that the DYRK family is a critical molecular target of ID-8 in hESC. To confirm this, the inventors designed miRNA to target the DYRK family and evaluated them after transfection by immunoblotting (FIG. 4D-E). Next the inventors performed colonal proliferation assays with the stably transfected hESCs. The DYRK miRNA-transfected hESCs maintained their undifferentiated state as did the un-transfected or scrambled control miRNA-transfected cells in MEF-CM or on feeders, however, survival and self-renewal were enhanced in the DYRK-knocked down cells in the presence of Wnt ligand in clonal proliferation assays (FIG. 4F). The effect of DYRK-knockdown was very similar to ID-8 treatment in the clonal proliferation assay. Taken together, these data indicate that the DYRK family is a direct target of ID-8 and ID-8 enhances Wnt-mediated hESC survival and self-renewal inhibition of DYRKs.

### Example 9

The development of a xeno-free, feeder-free hESC culture system using ID-8 and Wnt activation was investigated. After extensive investigation, the inventors determined that the minimal media required to maintain hESC proliferation and pluripotency consisted of DMEM/F-12 with bFGF (4 ng/ml), Wnt3a (25 ng/ml), ID-8 (0.5 μM) and Insulin-Transferrin-Selenium (ITS). In the absence of ID8 no colonies were maintained. Next the inventors removed the bFGF from the medium. hESC still maintained their undifferentiated morphology however, proliferation was significantly diminished. The inventors next sought to replace the Wnt ligand with several small molecule GSK-3 inhibitors, BIO (11), CHIR99021 (22) and Kenpaullone (23) in ID-8 containing medium with or without bFGF. The inventors found three conditions; 0.2 μM CHIR90021 plus bFGF, 1.0 μM Kenpaullone, 1.0 μM Kenpaullone plus bFGF, and 0.5 μM Kenpaullone plus bFGF could maintain hESCs. However, in contrast to Wnt ligand containing medium, hESCs became unstable after few passages, subsequently stabilized and maintained pluripotent cores were surrounded with spontaneously differentiated cells (FIG. 5). Under these conditions, manually selection of undifferentiated hESC colonies from the surrounding cells was required at every passage to maintain pluripotent cultures.

Initially, cells were maintained on Matrigel, a complex animal derived ECM. However, it would be advantageous to use a defined xeno-component-free ECM. The inventors found that a combination of Fibronectin-Laminin-Vitronection (FLV) could support hESC self-renewal and maintain undifferentiated colony morphology. Because of the extremely low level of protein in these cultures (i.e. no albumin and matrigel), the hESCs are extremely sensitive to damage caused by the dissociation enzymes normally used for passaging. ROCK inhibitor, which has been reported to enhance hESC survival (10), was not effective under these conditions and also induce differentiation, even with manual mechanical passaging. To develop a compatible passaging process, the inventors investigated several integrin antagonists. Both the disintegrin Echistatin and GRGDTP peptides effectively caused colony detachment and allowed for the passage of hESC in our medium.

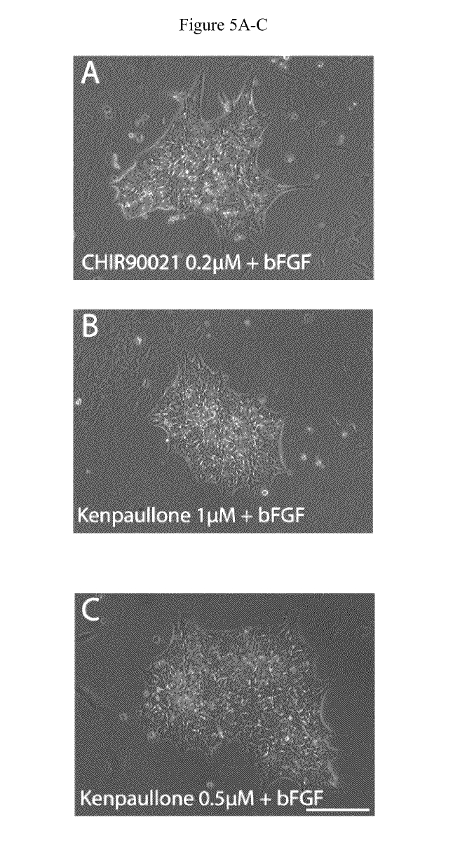

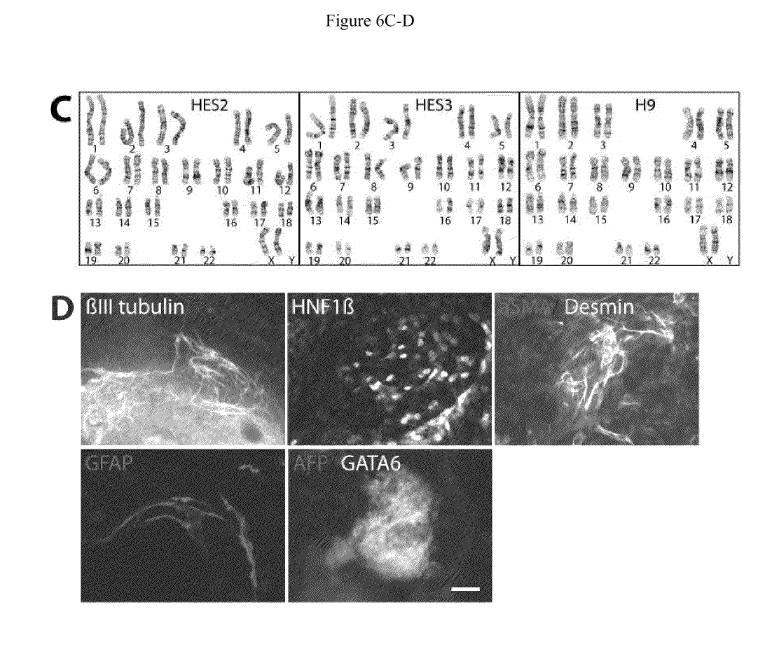

Combining these discoveries, a simple xeno-free and feeder-free culture system was developed incorporating ITS/Wnt/ID-8/DMEM/F-12 media, FLV-coated dishes and disintegrin passaging. hESCs transferred into this culture system from normal serum replacement-containing medium were stable and required no culture adaptation. All three hESC lines examined; HES2, HES3 and H9 maintained undifferentiated morphologies for >17 passages (FIG. 6A) in this system without bFGF supplementation or over 20 passages with bFGF. All pluripotent stem cells markers examined, including OCT4, SOX2, NANOG, SSEA3, SSEA4, GCTM-2, TRA-1-60, TRA-1-80 and Alkaline Phosphatase, were expressed in the cells (FIG. 6B). A normal karyotype was also maintained after 17-22 passages (FIG. 6C). After 17 passages, embryoid body-mediated in vitro differentiation and immunostaining for the three germ layer cell markers was performed to evaluate differentiation capacity of the cultured hESC (FIG. 6D). These cells readily differentiated into all three germ layers: with ectodermal cells expressing βIII-tubulin and GFAP, mesodermal cells expressing α-SMA and Desmin, endodermal cells expressing AFP and GATA6, and definitive endodermal cells expressing HNF10. In addition, the cells formed teratomas containing derivatives of all three germ layers when injected subcutaneously into immuno-deficient mouse after 22 passages (FIG. 6E). To preclude the possibility that the culture system could maintain the undifferentiated state only in female hESC, a male hESC line H1(1) and a human dermal fibroblast-derived iPSC (24) line were examined in our culture system. Both cell line cells were maintained in the undifferentiated state for at least 15 passages (FIG. 6A). These data suggest our xeno-free, feeder-free culture system can be utilized to maintain pluripotent cells long-term without losing differentiation ability and normal karyotype.

All references cited herein are incorporated by reference in their entirety as though fully set forth. Unless defined otherwise, technical and scientific terms used herein have the same meaning as commonly understood by one of ordinary skill in the art to which this invention belongs. Singleton et al., Dictionary of Microbiology and Molecular Biology 3rd ed., J. Wiley & Sons (New York, N.Y. 2001); March, Advanced Organic Chemistry Reactions, Mechanisms and Structure 5th ed., J. Wiley & Sons (New York, N.Y. 2001); and Sambrook and Russel, Molecular Cloning: A Laboratory Manual 3rd ed., Cold Spring Harbor Laboratory Press (Cold Spring Harbor, N.Y. 2001), provide one skilled in the art with a general guide to many of the terms used in the present application.

Many modifications and variations of the invention as hereinbefore set forth can be made without departing from the spirit and scope thereof and therefore only such limitations should be imposed as are indicated by the appended claims:

All patent and literature references cited in the present specification are hereby incorporated by reference in their entirety.

### REFERENCES

1. J. A. Thomson et al, Science 282, 1145 (Nov. 6, 1998).
2. B. E. Reubinoff, M. F. Pera, C. Y. Fong, A. Trounson, A. Bongso, Nat Biotechnol 18, 399 (Apr. 1, 2000).
3. M. Amit el al, Dev Biol 227, 271 (Nov. 15, 2000).
4. T. E. Ludwig et al, Nat Biotechnol 24, 185 (Mar. 1, 2006).
5. J. Lu, R. Hou, C. J. Booth, S.-H. Yang, M. Snyder, Proc Natl Acad Sci USA 103, 5688 (Apr. 11, 2006).
6. Y. Liu et al, Biochem Biophys Res Commun 346, 131 (Jul. 21, 2006).
7. S. Yao et al., Proc Natl Acad Sci USA 103, 6907 (May 2, 2006).
8. A. Swistowski et al. PLoS ONE 4, e6233 (Jul. 14, 2009).
9. K. Hasegawa, T. Fujioka, Y. Nakamura, N. Nakatsuji, H. Suemori, Stem Cells 24, 2649 (Dec. 1, 2006).
10. K. Watanabe et al., Nat Biotechnol 25, 681 (Jun. 27, 2007).
11. N. Sato, L. Meijer, L. Skaltsounis, P. Greengard, A. H. Brivanlou, Nat Med 10, 55 (Jan. 1, 2004).
12. G. Dravid et al., Stem Cells 23, 1489 (Nov. 1, 2005).
13. L. Cai et al., Cell Res 17, 62 (Jan. 1, 2007).
14. K. Melchior et al., Biological Chemistry 389, 897 (Jul. 1, 2008).
15. T. Sumi, N. Tsuneyoshi, N. Nakatsuji, H. Suemori, Development 135, 2969 (Sep. 1, 2008).
16. K. H. Emami et al., Proc Natl Acad Sci USA 101, 12682 (Aug. 24, 2004).
17. M. McMillan, M. Kahn, Drug Discov Today 10, 1467 (Nov. 1, 2005).
18. T. Miyabayashi et al., Proc Natl Acad Sci USA 104, 5668 (Mar. 27, 2007).
19. A. s. I. Lukaszewicz, M. K. McMillan, M. Kahn, Journal of medicinal chemistry (Jan. 4, 2010).
20. C. Xu et al., Nat Biotechnol 19, 971 (Oct. 1, 2001).
21. T. Miyabayashi, M. Yamamoto, A. Sato, S. Sakano, Y. Takahashi, Biosci. Biotechnol. Biochem. 72, 1242 (Jan. 1, 2008).
22. Q.-L. Ying et al., Nature 453, 519 (May 22, 2008).
23. C. A. Lyssiotis et al., Proc Natl Acad Sci USA 106, 8912 (Jun. 2, 2009).
24. K. Hasegawa, P. Zang, W. Lu, M. F. Pera, Stem Cells submitted.
25. M. J. Martin, A. Muotri, F. Gage, A. Varki, Nat Med 11, 228 (February, 2005).
26. C. Ellerström et al., Stem Cells 24, 2170 (Oct. 1, 2006).
27. K. Rajala et al., Hum Reprod 22, 1231 (May 1, 2007).
28. J. M. Crook et al., Cell Stem Cell 1, 490 (Nov. 15, 2007).
29. S. C. Desbordes et al., Cell Stem Cell 2, 602 (Jun. 5, 2008).
30. R. Damoiseaux, S. P. Sherman, J. A. Alva, C. Peterson, A. D. Pyle, Stem Cells 27, 533 (Mar. 1, 2009).
31. T. Lin et al., Nat Meth 6, 805 (Nov. 18, 2009).
32. V. I. Rebel et al, Proc Natl Acad Sci USA 99, 14789 (Nov. 12, 2002).
33. J.-L. Teo, H. Ma, C. Nguyen, C. Lam, M. Kahn, Proc Natl Acad Sci USA 102, 12171 (Aug. 23, 2005).
34. H. Ma, C. Nguyen, K.-S. Lee, M. Kahn, Oncogene 24, 3619 (May 19, 2005).
35. A. Marson et al., Cell Stem Cell 3, 132 (Aug. 7, 2008).
36. Willert K, et al. (2003) Nature 423:448-452.
37. Haegele L, Ingold et al. (2003) Mol Cell Neurosci 24:696-708.
38. Chenn A and Walsh C A (2002) Science 297:365-369.
E. Walsh, J. et al. (2003) APMIS, 111(1):197-211.
39. Amit et. Al. (2004) Biol. Reprod. 70: 837-845.
40. Richards et. Al. (2002) Nat. Biotech. 20: 933-936.

What is claimed is:

1. A culture medium for maintaining proliferation and pluripotency of human embryonic stem cells (hESCs), the culture comprising:
(a) a basal medium;
(b) Wnt3a protein; and
(c) a small molecule Wnt signaling modulator.

2. The culture medium according to claim 1, wherein the basal medium comprises DMEM/F-12.

3. The culture medium according to claim 1, wherein the small molecule Wnt signaling modulator is ID-8, IQ-1, ICG-427, BIO, LiCl, a combination thereof, a small molecule structurally related to ID-8, IQ-1, ICG-427, BIO, LiCl, or a pharmaceutical equivalent, analog, derivative, salt or prodrug of any of the foregoing or any molecule that modulates Wnt signaling in order to maintain hESCs in an undifferentiated, state.

4. The culture medium according to claim 1, wherein the culture medium further comprises Insulin-Transferrin-Selenium (ITS).

5. A method of culturing pluripotent human embryonic stem cells (hESCs), comprising:
(a) providing a quantity of hESCs;
(b) placing the quantity of hESCs in a culture vessel coated with an extracellular matrix; and
(c) culturing the quantity of hESCs with a culture medium comprising a basal medium, Wnt3a protein, and a Small molecule Wnt signaling modulator, wherein the hESCs remain pluripotent and proliferate;

6. The method according to claim 5, wherein the basal medium comprises DMEM/F-12.

7. The method according to claim 5, wherein the culture medium further comprises Insulin-Transferrin-Selenium (ITS).

8. The method according to claim 5, wherein at least a portion of the quantity of hESCs are maintained for more than 17 passages.

9. The method according to claim 5, wherein at least a portion of the quantity of hESCs can differentiate into all three germ layers after the culturing step.

10. A method of passaging a quantity of pluripotent human embryonic stem cells (hESCs), comprising:
(a) providing the quantity of hESCs in a first quantity of a culture medium comprising a basal medium, Wnt3 a protein, and a small molecule Wnt signaling modulator;
(b) adding a disintegrin and GRGDTP peptides to the quantity of hESCs;
(c) allowing the quantity of hESCs to detach from the dishes;
(d) rinsing the detached quantity of hESCs in a second quantity of the culture medium; and
(e) seeding at least a portion of the quantity of hESCs on new dishes.

11. The method according to claim 10, wherein the basal medium comprises DMEM/F12.

12. The method according to claim 10, wherein the small molecule Wnt signaling modulator is ID-8, IQ-1, ICG-427, BIO, LiCl, a combination thereof, a small molecule structurally related to ID-8, IQ-1, ICG-427, BIO, LiCl, or a pharmaceutical equivalent, analog, derivative, salt or prodrug of any of the foregoing or any molecule that modulates Wnt signaling in order to maintain hESCs in ah undifferentiated state

13. The method according to claim 10, wherein the culture medium further comprises Insulin-Transferrin-Selenium (ITS).

14. The method of claim 10, wherein the providing the quantity of hESCs further comprises providing hESCs On Fibronectin-Laminin-Vitronection (FLV)-coated dishes.

15. The method of claim 10, wherein the disintegrin is Echistatin.

16. The method of claim 10, wherein at least a portion of the quantity of hESCs cells are passaged by repeating steps (a) through (e).

17. The method of claim 16, wherein steps (a) through (e) are repeated more than 17 times.

18. The method of claim 10, wherein the hESCs can differentiate into all three germ layers after the seeding step.

19. The method of claim 10, wherein the seeding further comprises seeding in a 1 to 2 ratio.

20. A composition comprising human embryonic stem cells (hESCs) and a culture medium, comprising:
a quantity of hESCs; and
a culture medium, comprising a basal medium, Wnt3a protein, and a small molecule Wnt signaling modulator.

21. The composition of claim 20, wherein the basal medium comprises DMEM/F-12.

22. The composition of claim 20, wherein the small molecule Wnt signaling modulator is ID-8, IQ-1, ICG-427, BIO, LiCl, a combination thereof, a small molecule structurally related to ID-8, IQ-1, ICG-427, BIO, LiCl, or a pharmaceutical equivalent, analog, derivative, salt or prodrug of any of the foregoing or any molecule that modulates Wnt signaling in order to maintain hESCs in an undifferentiated state.

23. The composition of claim 20, wherein the culture medium further comprises Insulin-Transferrin-Selenium (ITS).
